(19) United States
(12) Patent Application Publication
(10) Pub. No.: US 2025/0268504 A1
(43) Pub. Date: Aug. 28, 2025

(54) NEURAL ELECTRODE MADE WITH SOFT POLYMERS AND RAPID PROTOTYPING TECHNIQUES

(71) Applicant: SCUOLA SUPERIORE DI STUDI UNIVERSITARI E DI PERFEZIONAMENTO SANT'ANNA, Pisa (IT)

(72) Inventors: Ciro ZINNO, Portici NA (IT); Alice GIANNOTTI, Pisa (IT); IIaria CEDROLA, Roma (IT); Eugenio REDOLFI RIVA, Pisa (IT); Silvestro MICERA, Firenze (IT); Filippo AGNESI, Milano (IT); Emanuele RIZZUTO, Roma (IT)

(21) Appl. No.: 18/878,837

(22) PCT Filed: Jul. 5, 2023
(86) PCT No.: PCT/IB2023/056964

§ 371 Date: Dec. 24, 2024

(30) Foreign Application Priority Data
Jul. 6, 2022 (IT) 102022000014269

(51) Int. Cl. A61B 5/279; A61B 5/00; A61B 5/266; A61B 5/268; A61N 1/05

(52) U.S. Cl. CPC A61B 5/279; A61B 5/266; A61B 5/268; A61B 5/6847; A61N 1/0556; A61B 2560/0468; A61B 2562/0209; A61B 2562/125; A61B 2562/164; A61B 2562/227

(57) ABSTRACT

The present invention relates to the field of implantable neural interface devices and, in particular, it refers to a new implantable polymeric neural electrode for extra- or intra-neural applications, as well as to a process for the simple and economically sustainable manufacture thereof.

### FIELD OF THE INVENTION

The present invention concerns the field of implantable neural interface devices and, in particular, refers to a new implantable neural electrode for extra- or intra-neural applications, as well as to a process for the simple and economically sustainable manufacture thereof.

### STATE OF THE ART

Implantable electrodes are medical devices that can be used as bioelectronic interfaces to monitor and/or stimulate various biological tissues in order to treat or diagnose various diseases. Numerous studies, over the last few years, have focused on the realization of neural interfaces capable of making communication with the peripheral nervous system possible and efficient, guaranteeing modulation and recording of its electrical signals.

Despite the significant technological advances achieved, the characteristics of implantable devices developed to date have not always been able to guarantee a satisfactory performance in terms of long-term biocompatibility and capacity of recording and stimulating the neural signal.

There are many examples, both in the literature and on the market, of devices that allow this type of communication, whose components can be made with different materials, often metals as regards the conductive components (X. Navarro et al. J. Peripher. Nerv. Syst., vol. 10, no. 3, pp. 229-258, 2005, doi: 10.1111/j.1085-9489.2005. 10303.x). However, the physical-chemical properties of the latter are very different from those of the nervous tissue with which they interface, and this often leads to adverse inflammatory reactions being established, with consequent encapsulation of the device by a veil of fibrotic tissue synthesized by the fibroblasts, in turn recalled by the macrophages (Ratner D. B. Science 2015, Vol. 7 Issue 272). All this contributes to an increase in the electrical impedance at the interface with the electrode and to the decrease in the signal-to-noise ratio, therefore to a drop in the performance of the electrode made.

It can therefore be understood the decisive technological need to remedy these problems, especially in view of a chronic implantation of the neural interface. With a view to minimizing the unwanted response mechanisms and increasing the long-term biocompatibility of the neural devices, the selection of materials that best respect the characteristics of the tissue with which they will interface (peripheral nerves) appears of fundamental importance.

There are two fundamental aspects of the concept of biocompatibility in the context of the development of neural interface devices. The term superficial biocompatibility refers, in particular, to the limitation of any biochemical reactions that may occur following the implantation of the device and that involve the adsorption of the extracellular proteins and the consequent recall of cells of the immune system, which subsequently give rise to the reaction to a foreign body. Structural biocompatibility, on the other hand, can be guaranteed by a correct evaluation of the mechanical properties of the materials used, for example the Young's module, in consideration of the fact that the electrode is in direct contact with the peripheral nerve. Furthermore, the shape of the device is also crucial, which must minimize the load applied to the tissue, avoiding tensions that could damage it in case of long-term implantation.

Nerve tissue, because of its particular chemical structure that guarantees a high water content, is characterized by low Young's modulus values (10 Pa<E<10 kPa, for peripheral nerves 100-500 kPa), and has, within an appropriate deformation limit, an elastic behaviour under stress. In contrast, the metallic materials that make up the vast majority of the electrode implants or devices developed to date are characterized by much higher Young's modulus values (E>1 GPa) and, when subjected to deformation, the yield strength is decidedly lower than that characteristic of the nervous system (usually <5%) (H. Yuk, et al. Chem. Soc. Rev., vol. 48, no. 6, pp. 1642-1667, 2019, doi: 10.1039/C8CS00595H).

In addition to the above, the most common techniques for the realization of neural electrodes, for example photolithography, envisage a series of processes that make the manufacturing process of such devices long and complex, reducing the ability to customize the characteristics of the final prototype. Using photolithography, it is also very difficult to process conductive materials other than metals.

In this context, it therefore appears still urgent the need to have available neural interface electrodes that can combine mechanical and chemical-physical properties such as to make communication with the nervous system possible and efficient, together with greater ease of manufacture and application.

### SUMMARY OF THE INVENTION

The object underlying the present invention is to provide an implantable neural electrode and a method for the realization thereof that allow to overcome the problems encountered in the known art, with particular reference to the reduced long-term surface and structural biocompatibility, as well as the complexity of the already existing manufacturing processes found. This problem is solved by a device according to claim 1. Preferred features of the present invention are the subject of the dependent claims.

The invention provides in particular an implantable neural electrode comprising a flexible main body configured to be at least partially disposed around or inserted within a target nerve, which main body comprises, in a preferred embodiment thereof, the following elements:
a first electrically insulating layer;
a second electrically insulating layer; and
an electrically conductive pattern interposed between said first and second layers;

wherein said pattern comprises a first portion, preferably at least partially outwardly exposed, configured to electrically come into contact with said target nerve, and a second portion, preferably at least partially outwardly exposed, configured to electrically connect said electrode to an external device.

The electrode developed by the present inventors consists of polymeric materials capable of ensuring a high degree of surface and structural biocompatibility with respect to systems known in the art. In particular, the first layer and the second layer of the electrode object of the invention comprise at least one polymeric material while the electrically conductive pattern comprises at least one conductive hydrogel or polymer.

In a particular embodiment of the invention, both the insulating layers and the conductive layer of the neural electrode are substantially made up of polymeric materials and do not contain inserts of metallic materials. In particular, the insulating layers may be made with elastomeric polymers, such as silicone elastomers like polydimethylsiloxane (PDMS), thermoplastic elastomers such as polyurethane (PU), or liquid crystal elastomers (ECL). The conductive component, on the other hand, can be made with conductive hydrogels or polymers, for example polystyrene sulfonate (PEDOT:PSS).

This particular combination of materials allows first of all to reduce the possibilities of post-surgical immune and inflammatory response compared to the materials of traditional use for the bioelectronic devices. Furthermore, the use of conductive polymers instead of metals for the realization of the conductive pattern of the electrode also confers advantages from the point of view of the electrochemical performance of the device. Having the ability to swell upon contact with the biological fluids and being processed to assume the three-dimensional lattice conformation, a conductive hydrogel or polymer has a higher surface/volume ratio than a metal, a condition that results in an increase in the recording performance of the neural signal (thanks to the increased ion permeability) and a better ability to store charge. The particular structural conformation of the conductive polymer network allows a better ionic conductivity compared to metals, which therefore allows to improve the recording of the electrical nerve signals.

In a particular embodiment according to the invention, the insulating layers of the neural electrode comprise or substantially consist of one or more biodegradable polymeric materials, for example selected from polypeptides, proteins, such as fibroin, biodegradable elastomeric polymers, in particular polyglycerol sebacate (PGS), or gel or hydrogel of a gelling agent of natural origin, for example alginate or gelatin gel. The use of biodegradable polymeric materials offers the possibility of being able to control and modulate the capacity and/or the degradation times of the implanted device once the therapeutic function thereof is over. This translates into two important advantages: the first is represented by the possibility of avoiding a second surgical implant to explant the device once it has exhausted its function or in case of generation of a copious inflammatory reaction that could cause damage to the surrounding tissues; the second is represented by a greater “environmental sustainability”, with a considerable reduction of the problems and critical issues related to the disposal of the aforesaid devices. To date, tens of millions of tons of e-waste (about 50 million in 2018) are produced globally. Few of the latter are recycled, weighing heavily on the environmental health of a Planet that is already greatly compromised by the problems of plastic pollution and global warming.

While the selected biochemical characteristics are necessary to ensure the surface biocompatibility of the device, the mechanical ones are fundamental in order to respect its structural biocompatibility characteristic. In this regard, the choice of the polymeric materials mentioned above moves exactly to this direction, since these are in fact characterized by lower Young's Modulus values than those traditionally used in the sector.

A further important advantage of the device object of the invention is represented by the ability to minimize the mechanical mismatch with the nerve tissue with which it is placed into contact, consequently limiting any damage that could arise at the level of the nerve tissue. As already mentioned, the polymeric materials used for the realization of the device have a mechanical behaviour more similar to that of the biological tissues and are characterized by low Young's modulus values thanks to the high percentage of water they contain. The particular combination of soft, flexible and stretchable materials, allows in particular the device object of the invention to adapt to the nerve at which it is implanted, following it in its micro and macroscopic movements.

Advantageously, the electrode object of the invention can be made by 3D printing, through a 20 rapid process that allows to customize the final design. Preferably, the insulating and conductive components of the electrode are made using polymeric compositions suitable for being printed, i.e. materials that possess rheological properties similar to those of inks, i.e. a viscosity that decreases with increasing deformation speed, shear thinning (H. Yuk et al., Nat. Commun., vol. 11, no. 1, Art. no. 1, March 2020, doi: 10.1038/s41467-020-15316-7). According to some aspects, the methodologies developed by the inventors provide for the possibility of also employing any solutions of conductive metallic nanostructures, but only in concentrations such as not to modify their optimal rheological and mechanical properties. As mentioned above, the opportunity to be able to realize 3D conductive structures provides a significant advantage in the field of the manufacture of neural electrodes, since this structure guarantees a high surface/volume ratio, increasing the performance of the devices in terms of signal recording.

Once a digitized model has been created for printing using Computer Aided Design (CAD), the manufacturing process developed by the inventors consists of a single step, i.e. printing the three-dimensional file previously designed and imported into the software of the 3D printing device. Unlike the technologies known in the art, the process developed by the inventors therefore does not require further steps or additional materials, such as, for example, photo-initiating agents used in the photolithography techniques. Furthermore, contrary to the specific photolithographic processes used for the manufacture of the devices of the known art, which generally envisage using different machines, the process object of the invention does not provide for the produced electrode to have to be physically moved from one machine to another; the manufacture is in fact reduced to a single process, reducing the risk of introducing contaminations into the system.

In particular, the manufacturing process object of the present invention does not require the execution of a step of alignment with an external structure or the use of moulds (e.g. mold/printed-circuit-board, PCB), preventing errors in the manufacturing step or contaminations due to the physical movement of the device from one machine to another.

All this makes it possible to greatly minimize the manufacturing times and complexity compared to the traditional methods, which generally envisage aligning different structures with a mm/μm accuracy, which entails difficulties from the point of view of the manufacture itself and from the point of view of manufacturing time, which will inevitably be lengthened.

The possibility of customizing the characteristics of the final device through the design of the CAD printing model also allows to give the latter complex geometries, if necessary, as well as to define specific internal patterns that can best influence its mechanical properties. Using the same manufacturing process, it is possible to realize an extraneural device, intended to be wound around the nerve, or an intraneural device, intended to be inserted within the nerve, so as to reach the internal fascicles of the same, and greatly increase the recording/stimulation selectivity. The ease of customization of the device is also an advantage from the point of view of the design of the active sites of the electrode, i.e. the regions where the passage of electric current from the nerve to the electrode takes place and vice versa. These, in fact, can be suitably disposed along the electrode following a histological analysis of the nerve, in order to identify the number of fascicles present inside it and their arrangement. This makes possible a selective recording/stimulation of only the fascicles of interest of the target nerve of the application.

These characteristics of versatility of the proposed manufacturing process constitute fundamental technological advantages compared to the traditional approaches, allowing to obtain devices not only more biocompatible and customizable, but also to substantially reduce their times and costs of realization.

The following are also the object of the present invention:
a system comprising an electrode according to any one of the embodiments described herein operatively connected to an external device by means of said at least one second portion of the electrically conductive pattern;
a process for the manufacturing of an implantable neural electrode according to any one of the embodiments illustrated in the present description and in the claims.

Other advantages and features of the present invention will become apparent from the following detailed description.

### BRIEF DESCRIPTION OF THE DRAWINGS

FIG. 1. FIG. 1 shows a perspective view (above) and three schematized cross-sections of a neural electrode according to a first embodiment of the present invention. The section indicated with the number 1, where the silver glue is present, represents the part of the electrode where the connections with the external electronics are made. The section indicated with the number 2 represents the part where the conductive pattern is isolated. The section indicated with the number 3 represents the sensitive part (active site), in which some portions of the conductive pattern are exposed and the charge exchange between the electrode and the target nerve is possible.

FIG. 2. FIG. 2 shows in schematized form a process of depositing silver glue on exposed conductive portions of an electrode according to the embodiment illustrated in FIG. 1, and aligning a custom printed circuit board to connect the device to external electronics.

FIG. 3. FIG. 3 shows (a) an extraneural electrode according to an embodiment of the present invention implanted around the nerve; and (b) an intraneural electrode according to an embodiment of the present invention implanted within the nerve.

FIG. 4. Connection diagram of an electrode according to any embodiment of the present invention for neural recording and stimulation application.

FIG. 5. Prototype of soft cuff type electrode printed with rapid prototyping techniques according to an embodiment of the present invention.

FIG. 6. Apparent viscosity of the compositions or conductive inks used for the manufacture of an electrode according to the present invention, as the formulations vary.

FIG. 7. Young's module of the PDMS insulating layer made with different combinations of printing parameters according to the present invention.

### GLOSSARY

The terms employed in the present description are as generally understood by the person skilled in the art, unless otherwise indicated.

As used in the context of the present invention, the term “neural electrode” refers to an electrode intended to be electrically interfaced with the nervous system of a subject in need thereof, in particular the central nervous system, the peripheral nervous system, the autonomic nervous system and/or the enteric nervous system, in particular with one or more nerves and/or neural fibres. The term “nerve” can refer to one or more fibres of the nervous system that use electrical and chemical signals to transmit information. For example, in the peripheral nervous system a nerve can transmit motor, sensory, and/or enteric information from one part of the body to another.

The expression “compositions having pseudo-plastic and/or thixotropic properties” refers, in the context of the invention, to compositions exhibiting shear thinning properties, i.e. a reduction in the viscosity of the fluid with increasing deformation speed.

The term “biocompatible” in the context of the present invention means any material suitable for interfacing with biological systems, whether living tissues, microorganisms or organisms, i.e. which does not have any toxic or harmful effect on such systems. In particular, by the term “biocompatible” is meant the ability of any material exemplified in the present description to be metabolized, incorporated or supplemented by living organisms without any deleterious effect on their vital functions.

By the term “biodegradable” is meant, in the context of the present invention, any material capable of degrading over time by the action of enzymes, by hydrolytic action, by microbial action and/or by other similar mechanisms in the human body. As used in the context of the present invention, the term “biodegradable” also comprises materials that are degradable but are not necessarily absorbed into or by the human body.

At any point of this description or of the claims, the term “biodegradable” may be replaced by the term “biocorrodible” or “bioabsorbable” or “bioresorbable” or “biodissolvable”.

In the context of the present description, the expression “operatively connected” and the like reflects a functional relationship between the various components of an electrode or system according to the present invention, i.e. it means that such components are correlated so as to perform a designated function. The “designated function” may vary depending on the different components involved in the connection; for example, the designated function of an electrode operatively connected to an external device such as a neural electrostimulator is to provide electrical current to a nerve to electrically stimulate it. A person skilled in the art would readily understand and identify the designated functions of each individual component of the electrode or of the system of the invention, as well as the connections therebetween, based on the teachings of the present description.

In the context of the invention, the term “flexible” refers to the ability of the main body of an electrode according to any one of the embodiments described herein to conform to the contours of the target nerve on which or within which it is positioned. The flexibility and elasticity of the main body of the electrode according to the invention is given by the materials of which it is substantially composed as illustrated in the detailed description.

By the expression “electrically conductive” is meant, in the context of the invention, a material having a conductivity preferably at least greater than 15 S/cm. By the expression “electrically insulating” is meant, in the context of the invention, a material(s) having a conductivity preferably not greater than 10-16 S/cm.

By the term “subject” is meant, in the context of the present invention, preferably a mammal, in particular a human being.

In the context of the present description, the expressions “active sites” or “sensitive sites” are used to identify the portions 31 of the electrically conductive pattern 20 of an electrode according to any one of the variants described herein, which are configured to electrically come into contact with a target nerve.

At any point of the description and of the claims, the term “comprising” may be replaced by “consisting of”.

### DETAILED DESCRIPTION OF THE INVENTION

Various embodiments and variants of the invention and parts thereof, based on different aspects thereof which can be used separately or in combination, will be described below.

Analogous components are denoted in the different Figures with the same reference numeral. In the detailed description below, further embodiments and variants with respect to embodiments and variants already discussed in the same description will be illustrated limitedly to the differences with what is already stated.

Furthermore, as mentioned, the different embodiments and variants described below are likely to be employed in combination.

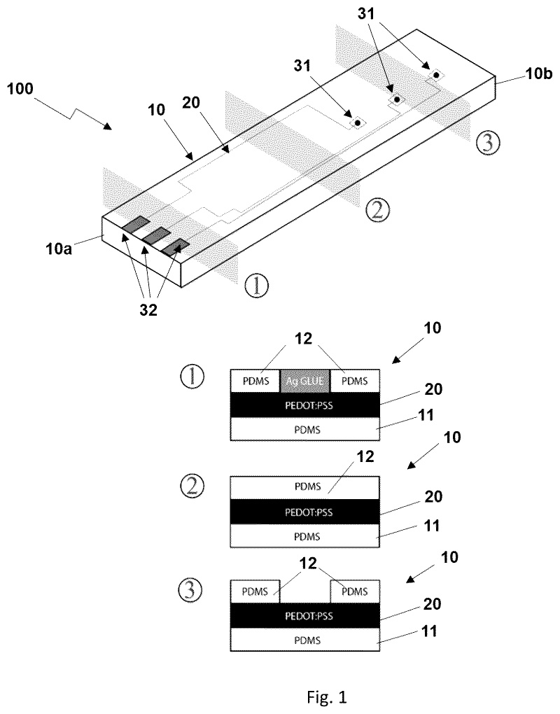

With reference initially to FIG. 1, an implantable neural electrode according to a preferred embodiment of the invention is collectively denoted 100.

The implantable neural electrode 100 according to the invention comprises a flexible main body 10 extending between a first end 10a and a second end 10b, configured to be at least partially disposed around or inserted within a target nerve, for example to modulate and/or record electrical signals thereof.

To this end, the main body 10 of an electrode according to any one of the embodiments described herein comprises:
a first electrically insulating layer 11;
a second electrically insulating layer 12; and
an electrically conductive pattern 20 interposed or positioned between said first and second insulating layers.

In the context of the present invention, by the term “pattern” is meant a predefined pattern of electrically conductive material made in such a way as to define one or more conductive traces or conductive circuit intended to electrically connect the electrode 100 to a target nerve and/or to an external device. The electrically conductive pattern according to the present description is therefore used to connect and/or close an electrical circuit and thus performs the function of electrical connector or “electrical interconnection”.

In particular, the pattern 20 of an electrode according to any one of the embodiments illustrated in the present description and in the claims comprises at least one first portion 31, preferably outwardly exposed, configured to electrically come into contact with said target nerve, and at least one second portion 32, preferably outwardly exposed, configured to electrically connect said electrode to an external device.

By the expression “outwardly exposed” is meant, in the context of the present description, that said portion 31 or 32 is not covered or coated by the second layer 12 of the main body of the electrode, but is exposed in such a way as to be able to come into direct contact with the surface of said target nerve or with an external device, respectively, i.e. to connect electrically with said target nerve or external device.

Advantageously, the first electrically insulating layer 11 and the second electrically insulating layer 12 of the electrode 100 comprise at least one “soft” polymer, in particular at least one elastomeric polymer, while the pattern 20 of the electrically conductive electrode 100 comprises at least one conductive hydrogel or polymer.

As will be illustrated in detail below, according to one aspect of the invention, the first layer 11, the second layer 12 and the pattern 20 of an implantable neural electrode as described herein are produced from compositions having pseudo-elastic and/or thixotropic properties. Said compositions have in particular shear thinning properties, i.e. a viscosity that decreases with increasing deformation speed; in other words, said compositions have suitable rheological properties such that they can be subjected to a printing process. According to a preferred aspect, the first layer 11, the second layer 12 and the pattern 20 of an electrode according to the invention are obtained or prepared using a printing technique, in particular they are prepared from said compositions, for example the compositions illustrated below in the present description, using a printing technique. To this end, it is possible to employ any printing technique known in the sector, preferably an extrusion printing technique, as will be illustrated in detail below in the present description.

In preferred embodiments of the invention, the first layer 11 and the second layer 12 of the electrode 100 comprise or substantially consist of polymeric material, in particular “soft” polymeric material, or combinations of many soft polymeric materials. Preferably, said first layer 11 and second layer 12 of the electrode 100 are substantially made of biocompatible and/or biodegradable polymeric material.

According to one aspect of the present invention, the first insulating layer 11 and the second insulating layer 12 of the electrode 100 substantially consist of one or more elastomeric polymers. By the term “substantially consist(s) of” is meant in the context of the present invention, that said first layer 11 and said second layer 12 consist of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one elastomeric polymer according to any one of the variants illustrated in the present description and in the claims.

According to a particularly preferred aspect of the present invention, the first insulating layer 11 and the second insulating layer 12 of the electrode 100 substantially consist of one or more biodegradable polymeric materials selected from polypeptides, proteins, elastomeric polymers, gels or hydrogels of a gelling agent of natural origin, and mixtures thereof.

By the term “substantially consist(s) of” is meant in the context of the present invention, that said first layer 11 and said second layer 12 consist of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one biodegradable polymeric material according to any one of the variants illustrated in the present description and in the claims. As will be explained in more detail below, by employing said one or more biodegradable polymeric materials it is possible to realize a three-dimensional electrode 100, i.e. a 3D neural interface, completely polymeric and biodegradable, with mechanical properties and degradation time adaptable to the different therapeutic needs.

According to a further aspect, the pattern 20 of an electrode according to any one of the embodiments described herein consists substantially of polymeric material, in particular of one or more conductive hydrogels or polymers.

By the term “substantially consist(s) of” is meant in the context of the present invention, that said pattern 20 consists of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one conductive hydrogel or polymer according to any one of the variants illustrated in the present description and in the claims.

A preferred embodiment of the invention refers in particular to an electrode 100 according to any one of the variants described herein, wherein said layer 11 and said layer 12 consist of at least 80%, at least 90%, at least 95% or at least 98% by weight of said at least one elastomeric polymer and wherein said pattern 20 consists of at least 80%, at least 90%, at least 95% or at least 98% by weight of said at least one conductive hydrogel or polymer.

A particularly preferred embodiment of the invention refers to an electrode 100 according to any one of the variants described herein, wherein said insulating layer 11 and said insulating layer 12 consist of at least 80%, at least 90%, at least 95% or at least 98% by weight of said at least one biodegradable polymeric material selected from any one of the biodegradable polymeric materials illustrated in the present description or mixtures thereof, and wherein said pattern 20 consists of at least 80%, at least 90%, at least 95% or at least 98% by weight of said at least one conductive hydrogel or polymer.

According to one aspect, any elastomeric polymer known to a person skilled in the art may be used for the realization of the insulating layers of the electrode according to the invention, in particular a biocompatible and/or biodegradable elastomeric polymer.

Said at least one elastomeric polymer is preferably selected from silicone elastomers, thermoplastic elastomers, liquid crystal elastomers (LCE), elastomeric polyesters and polycarbonates, and mixtures thereof. Examples of silicone elastomers include, but are not limited to, urea-based silicone copolymers, oxamide-based silicone copolymers, amide-based silicone copolymers, urethane-based silicone copolymers and mixtures thereof, in particular polydimethylsiloxane (PDMS) or polymethylvinylsiloxane or copolymers thereof. A thermoplastic elastomer may be at least one thermoplastic elastomer selected from elastomers based on styrene, olefin, vinyl chloride, urethane, polyester, polyamide, fluorine, conjugated butadiene and silicone. These include, for example, polyurethane (PU), polyolefins, ethylene-vinylacetate copolymers, or ethylene-containing rubbers.

According to a preferred aspect of the invention, said at least one elastomeric polymer is selected from PDMS, PU, poly--caprolactone (PCL), poly(glycolide-co-epsilon-caprolactone) (PGACL), poly(lactic acid-co-caprolactone) (PLACL), copolymers thereof and/or mixtures thereof, even more preferably said at least one elastomeric polymer is PDMS or a copolymer of PGACL/HDI (hexamethylene diisocyanate)/PLACL.

As a person skilled in the art knows, an elastomeric polymeric material possesses high flexibility and elongation at break and can be rendered biodegradable if monomers with chemical groups (such as esters, starches and carbonates) are introduced which can be cleaved by acid hydrolysis or enzymatic hydrolysis.

Examples of biodegradable elastomeric polymers that can be employed for the realization of the insulating layers of the electrode according to the invention include polyhydroxyalkanoates (PHA), in particular poly(3-hydroxybutyrate), poly(4-hydroxybutyrate), poly(3-hydroxyvalerate), poly(3-hydroxyhexanoate), poly(3-hydroxyoctanoate), poly(3-hydroxydecanoate), PU, in particular branched chain PU, copolymers thereof and/or mixtures thereof.

According to a particularly preferred aspect, said at least one elastomeric polymer is a biodegradable elastomeric polymer selected from PHA, PU, copolymers thereof and/or mixtures thereof, even more preferably said at least one elastomeric polymer is a biodegradable elastomeric polymer selected from poly(3-hydroxybutyrate), poly(4-hydroxybutyrate), poly(3-hydroxyvalerate), poly(3-hydroxyhexanoate), poly(3-hydroxyoctanoate), poly(3-hydroxydecanoate), polyglycerol sebacate (PGS), copolymers thereof and/or mixtures thereof, even more preferably is PGS or is a mixture of PGS and PCL.

As a person skilled in the art knows, PGS is obtained by polycondensation of glycerol and sebacic acid. The raw materials glycerol and sebacic acid are endogenous metabolites of the human body and have been widely used in the food, biomedical, and cosmetic industries. Advantageously, the PGS can be prepared without resorting to the use of any additive or catalyst in the polymerization process, eliminating the potential toxicity of the in vivo application. PGS elastomers are typically prepared in two steps: the first step envisages the synthesis of prepolymer through the polycondensation of glycerol and sebacic acid, the second step envisages cross-linking. The resulting PGS elastomers are transparent, almost colourless with high elasticity and programmable biodegradation. The PGS elastomers are designed to mimic the properties of elastin by building 3D lattice networks in the material. By varying the percentage of monomers and the reaction time, a modulation of the mechanical properties and degradation can be carried out, to adapt the resulting material to the various needs. For example, as mentioned above, the PGS can be combined with poly-caprolactone and then extruded by 3D printing.

In a further embodiment of the invention, the insulating layers of the electrode according to any one of the variants described herein comprise or substantially consist of any biodegradable polypeptide known to a person skilled in the art. In a particularly preferred embodiment according to the present invention, said insulating layer 11 and said insulating layer 12 comprise or substantially consist of fibroin.

The fibroin extracted from silk has good properties of biocompatibility, biodegradability and excellent mechanical characteristics, which characteristics are well known in the technological field. In particular, fibroin can be treated with vacuum annealing cycles whose objective is to slow down the degradation process. The use of fibroin for the realization of the insulating layers of the electrode therefore allows to be able to control in the laboratory the life time required by the device and schedule the annealing procedure in order to modulate the biodegradation properties thereof.

In a further embodiment according to the present invention, said layer 11 and said layer 12 of the electrode comprise or substantially consist of a gel or hydrogel of a gelling agent of natural origin, in particular an alginate or gelatin gel of vegetable or animal origin. The latter may be extruded by any 3D printing device known to a person skilled in the art. The fundamental difference between these gels and the biodegradable elastomers is that gels are much softer and more flexible, but can withstand less stress and strain. These materials are therefore very interesting for applications of the electrode in body districts for which compliance and softness represent characteristics necessary for an adequate interface with the tissue, for example the cerebral cortex.

For the realization of the electrically conductive pattern of an electrode according to the invention use can be made of any conductive hydrogel or polymer known to a person skilled in the art, in particular a biocompatible conductive hydrogel or polymer. In particular, according to one embodiment, said at least one conductive hydrogel or polymer is selected from polyaniline, polypyrrole, poly(3,4-ethylenedioxythiophene) polystyrene sulfonate (PEDOT:PSS) and mixtures thereof, preferably said at least one conductive polymer is PEDOT:PSS.

According to one aspect of the invention, said conductive hydrogel is a hydrogel comprising a mixture of an elastomer and a conductive polymer preferably selected from the compounds listed above. Alternatively, said conductive hydrogel is a mixture of an elastomer and a solution of conductive metallic nanostructures, for example Au and/or Ag-based nanostructures, in concentrations such as not to alter the rheological and mechanical properties of the starting material employed for the realization of the pattern.

A preferred embodiment according to the present invention refers in particular to a neural electrode according to any one of the variants described herein, wherein said at least one elastomeric polymer is PDMS and wherein said at least one conductive polymer is PEDOT:PSS.

Preferably, the first layer 11, the second layer 12 and the pattern 20 of the electrode do not contain metallic nanostructures, components and/or inserts. In other words, according to one aspect of the invention, the electrode 100 is completely free of metallic nanostructures, components and/or inserts. In the context of the present invention the terms metallic nanostructures, components and/or inserts preferably comprise, but are not limited to, metallic nanostructures, components or inserts such as Au, Ag, Pt, Al, Cu, Pt-Ir, Ir and the like. Advantageously, the absence of these materials in the electrode of the invention allows to reduce, or even completely eliminate the occurrence of post-neural implantation inflammatory reactions that can be found with the devices of traditional use in the sector.

In a preferred embodiment according to the invention, each layer of the main body 10 of an electrode according to any one of the variants described herein, including the electrically conductive pattern 20, contains exclusively non-toxic materials, i.e. biocompatible materials.

In some alternative embodiments, the first layer 11, the second layer 12 and/or the pattern 20 of an electrode according to the invention may however contain a certain amount of said metallic nanostructures, components and/or inserts, for example Au and/or Ag nanostructures, provided that the presence of said nanostructures, components and/or inserts in the compositions from which said layers and/or patterns are produced does not alter the rheological, electrical and/or mechanical properties, in particular does not alter the pseudo-plastic and/or thixotropic properties, as well as the biocompatibility of the resulting material.

In certain aspects, the first layer 11, the second layer 12, and/or the pattern 20 of an electrode according to any one of the embodiments described herein further comprise at least one compound of choice among thickening agents, surfactants, solvents, and plasticizers.

In one embodiment, the pattern 20 of the electrode comprises any additive substance that allows to improve the conductivity thereof, in particular a surfactant and even more preferably an ionic surfactant. The above additive substances can be divided into materials composed of small molecules or polymers. Possible examples of materials composed of small molecules (including surfactants) include dimethyl sulfoxide (DMSO), Triton X100, fluorine-based surfactants such as Zonyl, glycerol, 1-butyl-3-methylimidazolium hexafluorophosphate, 4-(3-butyl-1-imidazolium)-1-butanesulfones. Important characteristics of such additive substances are represented by solubility in the above conductive polymer, and presence of highly acidic anions capable of behaving as dopants. Non-limiting examples of polymers that can be used as additives according to the present invention are represented by polyethylene glycol (peg), polyethylene oxide (PEO), polyvinyl alcohol (PVA) and copolymers thereof. In general, to obtain better elasticity and conductivity, sulfonate or sulfamide groups must be present within these materials.

The layers 11, 12 and the pattern 20 of an electrode according to any one of the embodiments described herein may also comprise traces of solvents, photoinitiating agents, or precursor compounds/agents employed for their preparation, as exemplified hereinafter in the present description.

As previously mentioned, the pattern 20 of an electrode according to any one of the embodiments described herein is configured not only to allow the electrode to be electrically interfaced to a target nerve but also to connect the electrode to at least one external device, in particular any electronic system known in the art for neural monitoring, stimulation and/or treatment.

According to one aspect of the invention, said pattern 20 comprises a plurality of said first portions 31 exposed at least partially outwardly and configured to electrically come into contact with said target nerve. In other words, as also illustrated by way of example in FIG. 1, the second insulating layer 12 of the electrode covers or coats the pattern 20 of the electrode in such a way as to leave outwardly exposed a plurality of portions 31 of said pattern, preferably in the form of points or elements of substantially circular or square shape. As mentioned, said exposed portions 31 represent the active or sensitive sites of the electrode 100, intended to come into electrical contact with the target nerve.

Preferably, the outwardly exposed portions 31 of the pattern 20 are positioned or disposed along the entire length of the main body 10 of the electrode 100, i.e. along a longitudinal direction L of main development of the electrode 100.

Based on a histological analysis of said target nerve, for example based on the number of fascicles present in its inside and their arrangement, a person skilled in the art will be able to determine the number of portions 31 of the pattern 20 of an electrode according to any one of the embodiments described herein, which is suitable to guarantee an optimal electrical connection with said target nerve. It is preferable that the electrode 100 according to any one of the embodiments described herein, comprises at least 2, at least 3, at least 4, at least 5 or at least 8 exposed outwardly portions 31 of the pattern 20 so as to be able to electrically come into contact with said target nerve. The number of active sites, i.e. the number of outwardly exposed portions 31 of the pattern 20, is dependent on the application and on the target nerve considered, and, according to a preferred aspect of the invention, is calculated based on the histology of the nerve of interest, in order to increase the stimulation/recording selectivity.

According to one aspect of the present invention, the pattern 20 of an electrode according to any one of the variants described herein, has at least one second exposed outwardly portion 32 and located at one end of the main body 10, so as to allow and facilitate the electrical connection between the electrode 100 and at least one external device.

Preferably, the pattern 20 of an electrode 100 according to the invention comprises at least a number “X” of outwardly exposed portions 32, preferably located at one end of the main body 10, where “X” represents the number of active sites of the electrode 100, i.e. the number of active sites of the pattern 20 of the electrode, e.g. calculated based on the histology of the target nerve of interest.

According to a preferred aspect of the invention, the pattern 20 of an electrode 100 according to any one of the embodiments described herein comprises at least 2, at least 3, at least 4, at least 5 or at least 8 outwardly exposed portions 32 of the pattern 20, preferably located at one end of the main body 10.

The pattern 20 of an electrode 100 according to any one of the embodiments described herein is preferably predefined based on a digitally processed pattern/design.

According to one aspect of the invention, said pattern 20 is preferably made in the form of strips, lines and/or points connected to each other.

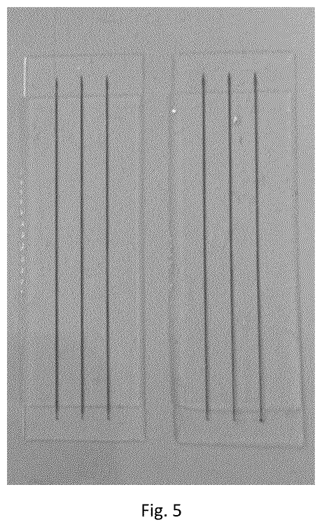

In a preferred embodiment of the electrode, said pattern 20 is made in the form of spaced apart parallel strips, in particular at least three parallel strips, as illustrated by way of example in FIG. 5. According to one aspect of the invention, said strips each have a width comprised between 50 and 500 μm, preferably equal to 150 μm and a length comprised between 10 and 200 mm, preferably equal to 45 mm.

According to one aspect of the invention, the main body 10 of an electrode according to any one of the embodiments described herein has a thickness in the order of nanometres, micrometres or millimetres. In some embodiments, said main body 10 has a thickness comprised between 50 nm and 10 mm, for example between 50 nm and 5 mm, between 50 nm and 1 mm, between 50 nm and 500 nm, between 50 nm and 200 nm, between 50 nm and 150 nm, between 50 nm and 100 nm. Such dimensions are considered optimal within the scope of the present invention with regard to electrode manoeuvrability, elasticity and mechanical compliance of the device to the body tissues.

Preferably, the main body 10 of the electrode according to the invention has a thickness comprised between 0.01 and 1 mm, preferably equal to 0.15 mm.

According to one aspect of the invention, the first end 10a and the second end 10b of the main body 10 of an electrode according to any one of the variants described herein are engageable with each other so as to close and maintain the electrode 100 in a desired position. In particular, the first end 10a and the second end 10b of the main body 10 of an electrode according to the invention are able to adhere to each other so as to maintain the electrode 100 in a closed configuration around a target nerve thanks to the adhesion forces generated.

According to one aspect of the invention, the main body 10 of an electrode according to any one of the variants described herein carries a slot 40 at said first end 10a, which slot 40 is configured to receive the second end 10b of the main body 10 and allow sliding therein of said main body 10 so as to close and maintain the electrode 100 in a desired position, preferably maintaining it in a closed configuration around a target nerve.

According to one aspect of the invention, the neural electrode according to any one of the embodiments described herein is an extraneural electrode, i.e. configured to be wound around a target nerve, and/or is an intraneural electrode i.e. configured to be inserted, at least partially, within a target nerve of a subject in need thereof.

According to a further aspect of the invention, the main body 10 of an electrode according to any one of the embodiments described herein, in particular an extraneural electrode, is of the “cuff” type.

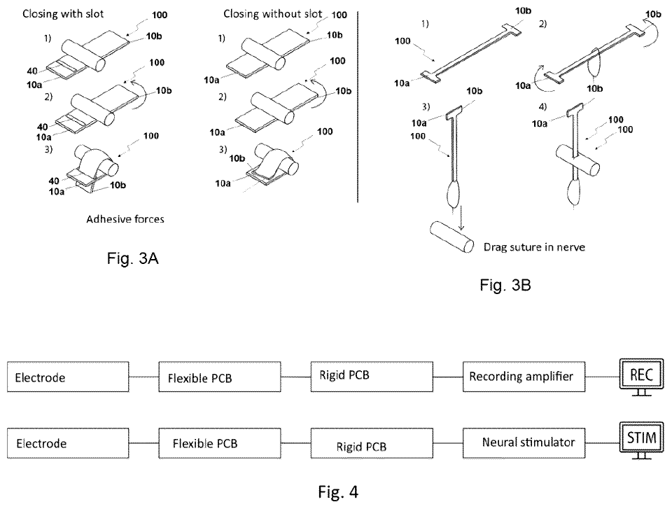

According to a further aspect of the invention, said electrode according to any one of the embodiments described herein is an intraneural electrode, in particular an intraneural electrode in which the first end 10a and the second end 10b of the main body 10 are wider than the part of said main body extending between the aforesaid ends, as shown purely by way of example in FIG. 3b.

This particular configuration allows the aforesaid ends to be connected to a flexible PCB, such as one of the PCBs previously exemplified in the present description, for example by depositing silver glue onto said one or more portions 32 of the conductive pattern of the electrode.

The main body 10 of an intraneural electrode according to any one of the embodiments described herein is configured to enter into a target nerve and comprises a conductive pattern 20 according to any one of the variants described above, having one or more exposed active sites, intended to come into electrical contact with said target nerve. The particular configuration illustrated in FIG. 3b is similar to what is referred to in the literature as a TIME electrode (Transversal Intrafascicular Multichannel Electrode).

According to one aspect of the invention, the intraneural electrode according to any one of the variants described herein is a TIME electrode.

According to one aspect of the invention, the main body 10 of an electrode according to any one of the embodiments described herein may also have a different configuration or geometry known in the art that is suitable for intraneural applications.

Preferably, said main body 10 of an electrode according to any one of the variants described herein has a length comprised between 5 and 250 mm, preferably equal to 50 mm, and a width comprised between 5 and 50 mm, preferably equal to 15 mm.

The present invention also relates to a system comprising an electrode according to any one of the embodiments described herein operatively connected to an external device by means of said at least one second portion 32 of the electrically conductive pattern 20 of the electrode.

Non-limiting examples of devices that may be connected to an electrode according to the present invention comprise a neural electrostimulator, an electrical amplification and/or recording system, a telemetry device, a syringe pump, a pressure pumping system, a haemostatic pumping system, a potentiostat, or combinations thereof.

According to a preferred aspect, the electrode 100 is operatively connected to said external device by means of at least one printed circuit board electrically connected to said at least one second portion 32 of the pattern 20 of the electrode, preferably a printed circuit board made of flexible polyimide.

Preferably, said at least one second portion 32 of the pattern 20 of the electrode 100 is electrically connected to said printed circuit board by means of silver glue.

According to a further aspect, the electrode 100 is operatively connected to said external device by means of a first flexible printed circuit board electrically connected to said at least one second portion 32 of the pattern 20 of the electrode, in turn connected to a second rigid printed circuit board, as illustrated purely by way of example in FIG. 4.

A further object of the present invention is represented by a process for the manufacturing of an implantable neural electrode 100 comprising a flexible main body 10 configured to be at least partially disposed around or inserted within a target nerve, said main body 10 extending between a first end 10a and a second end 10b, which process comprises the following steps:
(i) providing a first composition comprising at least one electrically insulating polymeric material or precursors thereof, and a second composition comprising at least one electrically conductive hydrogel or polymer or precursors thereof, wherein said compositions have pseudo-plastic and/or thixotropic properties;
(ii) depositing a layer of said first composition on a flat surface of a substrate, so as to form a first electrically insulating layer 11 of said main body 10 comprising said at least one polymeric material;
(iii) depositing a layer of said second composition onto the first layer 11 obtained in step (ii) so as to form, on said first layer, an electrically conductive pattern 20 comprising said at least one conductive hydrogel or polymer;
(iv) depositing a layer of said first composition onto the pattern 20 obtained in step (iii) so as to form, onto said pattern, a second electrically insulating layer 12 of said main body 10 comprising said at least one polymeric material, wherein said depositing is carried out in such a way that at least one first portion 31 and at least one second portion 32 of the pattern 20 are left at least partially outwardly exposed;
   wherein all depositing steps from (ii) to (iv) are performed by means of a printing technique.

According to one aspect of the invention, the first insulating layer 11 and the second insulating layer 12 of the electrode 100 substantially consist of polymeric material, preferably biocompatible and/or biodegradable, in particular of one or more elastomeric polymers. By the term “substantially consist(s) of” is meant in the context of the present invention, that said first layer 11 and said second layer 12 consist of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one elastomeric polymer according to any one of the variants described herein.

According to a particularly preferred aspect, the first insulating layer 11 and the second insulating layer 12 of the electrode 100 comprise or substantially consist of one or more biodegradable polymeric materials selected from polypeptides, proteins, elastomeric polymers, gels or hydrogels of a gelling agent of natural origin, and mixtures thereof. By the term “substantially consist(s) of” is meant in the context of the present invention, that said first layer 11 and said second layer 12 consist of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one biodegradable polymeric material according to any one of the variants illustrated in the present description and in the claims.

According to a further aspect, the pattern 20 of an electrode according to any one of the embodiments described herein consists substantially of polymeric material, in particular of one or more conductive hydrogels or polymers. By the term “substantially consist(s) of” is meant in the context of the present invention, that said pattern 20 consists of at least 70%, at least 75%, at least 80%, at least 85%, at least 90%, at least 91%, at least 92%, at least 93%, at least 94%, at least 95%, at least 96%, at least 97%, at least 98% or at least 99% by weight of said at least one conductive hydrogel or polymer.

In a preferred embodiment of the invention said layer 11 and said layer 12 consist of at least 80%, at least 90%, at least 95% or at least 98% by weight of said at least one elastomeric polymer and said pattern 20 consists of at least 80%, at least 90%, at least 95% or at least 98% by weight of said at least one conductive hydrogel or polymer.

Said at least one polymeric material and said at least one conductive hydrogel or polymer may be selected from any one of the polymers previously mentioned in the present description and in the claims. According to a preferred aspect, said at least one elastomeric polymer is selected from silicone elastomers, thermoplastic elastomers, liquid crystal elastomers, elastomeric polyesters and polycarbonates, and mixtures thereof, in particular is selected from PDMS, PU, PCL and mixtures thereof, even more preferably is PDMS.

According to a particularly preferred aspect, said at least one biodegradable polymeric material is a biodegradable elastomeric polymer selected from PHA, PU, copolymers thereof and/or mixtures thereof, even more preferably said at least one biodegradable elastomeric polymer is selected from poly(3-hydroxybutyrate), poly(4-hydroxybutyrate), poly(3-hydroxyvalerate), poly(3-hydroxyhexanoate), poly(3-hydroxyoctanoate), poly(3-hydroxydecanoate), PGS, copolymers thereof and/or mixtures thereof, preferably is PGS or is a mixture of PGS and PCL.

According to a further aspect, said at least one biodegradable polymeric material comprises or consists of any biodegradable polypeptide known to a person skilled in the art, preferably fibroin. According to a further aspect, said at least one biodegradable polymeric material comprises or consists of a gel or hydrogel of a gelling agent of natural origin, preferably is an alginate or gelatin gel of vegetable or animal origin.

In a preferred embodiment, the first composition provided in step (i) of a process according to the invention comprises precursors of said at least one polymeric material according to any one of the variants illustrated in the present description and in the claims i.e. one or more reagents suitable for obtaining the polymerization of the aforesaid material.

Purely by way of example, the first composition provided in step (i) of the process comprises PDMS precursors, wherein said precursors comprise PDMS monomers and at least one crosslinking agent; preferably the weight ratio between said monomers and said crosslinking agent is equal to 10:1.

According to one aspect of the invention, said at least one conductive polymer is selected from polyaniline, polypyrrole, poly(3,4-ethylenedioxythiophene) polystyrene sulfonate (PEDOT:PSS) and mixtures thereof, preferably is PEDOT:PSS.

In a preferred embodiment, the second composition provided in step (i) of a process according to the invention is a suspension of said at least one conductive polymer in a mixture of water and at least one organic solvent. Depending on the conductive polymer or the selected precursors thereof, a person skilled in the art will be able to select the most suitable solvent mixture for the solubilization or dispersion of said conductive polymer or precursors.

Preferably, the second composition provided in step (i) of a process according to the invention comprises at least one conductive polymer which has been subjected to a pretreatment by freeze-drying. Purely by way of example, said conductive polymer is frozen and subsequently freeze-dried for a predefined period of time, for example 72 hours. While on the one hand this improves the conductive properties of the material, bringing the polymer chains closer together and concentrating them more, on the other hand it ensures the subsequent formation of a stable 3D structure in water, capable of swelling.

According to a further aspect, a process according to any one of the embodiments described herein comprises at least one step, prior to said step (i) wherein said at least one conductive polymer is subjected to freeze-drying.

According to one aspect of the invention, said first and second compositions provided in step (i) of a process according to any one of the embodiments described herein may comprise at least one further substance or additive compound selected from thickening agents, surfactants, plasticizers, photoinitiating agents and solvents, for example selected from any one of the additive substances previously exemplified in the text of the present description. According to one aspect of the invention, the second composition provided in step (i) according to any one of the embodiments described herein comprises at least one agent known in the art which is capable of improving the conductivity of said conductive hydrogel or polymer, preferably a surfactant, even more preferably an ionic surfactant.

Alternatively, the second composition provided in step (i) of a method according to the present invention comprises precursors of an electrically conductive hydrogel and metallic nanostructures, such as Au and/or Ag nanostructures in an amount such as not to alter the pseudo-plastic and/or thixotropic properties of said composition, as well as the biocompatibility of the resulting pattern.

In a preferred embodiment of the process according to the invention, said first composition provided in step (i) comprises PDMS monomers and at least one crosslinking agent and said second composition comprises PEDOT:PSS dispersed in a solution of water and dimethyl sulfoxide (DMSO).

In a further preferred embodiment of the process according to the invention, said first composition provided in step (i) comprises PGS monomers and at least one crosslinking agent and said second composition comprises PEDOT:PSS dispersed in a solution of water and dimethyl sulfoxide (DMSO).

According to a preferred aspect of the invention, the first and second compositions provided in step (i) of the process according to the invention do not contain, i.e. are totally free of metallic nanostructures, components and/or inserts, such as, preferably, metallic nanostructures, components or inserts such as Au, Ag, Pt, Al, Cu, Pt-Ir, Ir and the like. Preferably, said compositions are non-toxic or biocompatible. The first and second compositions provided in step (i) of a process according to the present invention may be prepared according to any one of the techniques known to a person skilled in the art. On the basis of the polymers or their selected precursors, a person skilled in the art will in fact be able to identify the appropriate reagents, solvents, additives and quantities of such compounds in order to obtain compositions having the desired rheological and mechanical properties as illustrated in the present description and in the claims.

According to one aspect of the invention, prior to each depositing step (ii), (iii) or (iv), said first or second composition is inserted into a syringe suitable for loading the aforesaid compositions into a printing device.

As mentioned above, in steps (ii), (iii) and (iv) of a process according to the present invention, the depositing of the compositions is carried out by means of a printing technique. To this end, it is possible to use any printing technique and therefore any machine intended for carrying out this printing technique, known in the sector.

According to one aspect of the invention, said printing technique may be selected from extrusion printing, inkjet printing, screen printing, stereolithography, digital light processing or combinations thereof. Preferably, said printing technique is an extrusion printing technique. In case stereolithography or digital light processing is employed, said first and second compositions provided in step (i) will further comprise at least one photoinitiating agent.

According to one aspect of the invention, said steps (ii) and (iv) are carried out by extrusion printing at an extrusion speed comprised between 7.5 and 10 mm/s, and/or at a pressure comprised between 3 and 3.5 bar and/or at room temperature.

According to a further aspect, said step (iii) is carried out by extrusion printing at an extrusion speed comprised between 2 and 20 mm/s, preferably equal to 6 mm/s, and/or at a pressure comprised between 0.2 and 3 bar and/or at room temperature.

A substrate suitable for use in a process according to any one of the embodiments described herein is any substrate suitable for receiving at least one layer of said first and/or second composition. Examples of suitable substrates are represented by polyimide, polystyrene, polypropylene, plexiglass, polytetrafluoroethylene (PTFE), glass, silicon and smooth polymeric materials resistant up to 120° C., preferably resistant to commonly used solvents (e.g. acetone, ethanol, propanol).

Preferably, said substrate is heated to a temperature comprised between 25 and 120° C., even more preferably it is equal to 80° C. The heating of the printing substrate has a dual purpose: to allow the complete evaporation of the solvent possibly present in the compositions and to further increase the conductivity of the printed conductive pattern.

Heating the printing surface up to 80° C. ensures, in particular, a rapid polymerization of the printed material, thus creating a stable structure on which further layers can subsequently be added. As the selected material varies, the temperature can be changed to ensure a proper polymerization.

In order to allow the complete formation of said first and/or second layer, and in particular the complete polymerization of said at least one elastomeric polymer, as well as the stabilization of said at least one conductive hydrogel or polymer immediately after each depositing step by printing, it is preferable that the time elapsing between said step (ii) and said step (iii) and/or between said step (iii) and said step (iv) is at least equal to 1 minute.

Preferably, each of the depositing steps (ii), (iii), and (iv) of a process according to any one of the variants described herein does not comprise the use of moulds. This is made possible, in particular, thanks to the possibility of controlling the temperature of the substrate at a temperature such that the polymerization of the selected materials takes place rapidly, avoiding the detachment of the extruded material. According to one aspect of the invention, in step (iii) of a process according to any one of the embodiments described herein, said second composition is deposited on said first layer 11 so as to form a predefined electrically conductive pattern 20 on the basis of a model/design previously processed in digital format.

Preferably, in step (iii) of a process according to any one of the embodiments described herein, said second composition is deposited onto said first layer 11 so as to form a pattern 20 comprising one or more strips, lines and/or points connected to each other.

Alternatively, in said step (iii) of a process according to any one of the embodiments described herein, said second composition is deposited on said first layer 11 so as to form an electrically conductive pattern 20 comprising a plurality of strips parallel to each other, having dimensions according to any one of the embodiments previously exemplified in the present description.

According to one aspect of the invention, in step (iv) of a process according to any one of the embodiments described herein, said first composition is deposited on said pattern 20 so as to leave outwardly exposed a plurality of said first portions 31, preferably at least a number of said portions 31 equal to 3, equal to 4, equal to 5, equal to 6, equal to 8. As mentioned above, the number of portions 31 of the pattern 20 that are left outwardly exposed, is dependent on the application and on the target nerve considered, and, according to a preferred aspect of the invention, is calculated based on the histology of the nerve of interest, to increase the stimulation/recording selectivity.

According to one aspect of the invention, in step (iv) of a process according to any one of the embodiments described herein, said first composition is deposited on said pattern 20 so as to leave outwardly exposed at least one second portion 32 of said pattern at an end of said main body 10, preferably at least a number “X” of portions 32, preferably at an end of the main body 10, where “X” represents the number of active sites of the electrode 100, i.e. the number of active sites of the pattern 20 of the electrode, for example calculated based on the histology of the target nerve of interest as previously mentioned. It is preferable that the depositing steps (ii), (iii) and (iv) of a process according to any one of the variants described herein are carried out in such a way as to obtain a resulting main body having a thickness comprised between 0.01 and 1 mm, preferably equal to 0.15 mm.

According to a further aspect of the invention, a method according to any one of the embodiments described herein comprises a further step (v) of obtaining a slot 40 at the first end 10a of the main body 10 of the electrode, wherein said slot is configured to receive the second end 10b of the main body 10 and allow said main body 10 to slide in it so as to close and keep the electrode 100 in a desired position.

The process according to the invention may comprise a further step of electrically connecting said at least one second portion 32 of the pattern 20 to at least one external device. Said device may be selected from a printed circuit board, a neural electrostimulator, an electrical amplification and/or recording system, a telemetry device, a syringe pump, a pressure pumping system, a haemostatic pumping system or combinations thereof.

According to a preferred aspect, the process according to any one of the embodiments described herein does not comprise performing a step of alignment with the electrode 100 with an external device, in particular with a printed-circuit-board (PCB).

Preferably, the process according to any one of the embodiments described herein further comprises a step of designing by computer-aided-design (CAD) the geometry and the dimensions of the electrode to be produced by said printing technique.

The present invention also relates to an implantable neural electrode 100 obtainable by a process according to any one of the embodiments described herein.

A further aspect of the invention refers to the use of a neural electrode according to any one of the embodiments described herein as an interface of the nervous system of a subject in need thereof, in particular as a peripheral or central nervous system (CNS) nervous interface, or, in other words, as an interface between the peripheral and/or central nervous system and at least one external device for bidirectionally transducing recorded information and/or sending signals between the human body and a computer. In particular, a preferred aspect of the invention concerns the use of an electrode according to any one of the embodiments described herein as a fixed or removable implant configured to interface with a nerve for the purpose of detecting and/or electrically stimulating an electrical activity in a subject. Said electrode can be implanted extraneurally or intraneurally depending on the desired application.

Within the meaning of the present invention, a “fixed implant” defines an electrode according to any one of the embodiments described herein capable of residing in vivo in said subject without producing adverse biological reactions for prolonged periods of time, such as for example beyond 7 days. Again within the meaning of the present invention, a “removable implant” defines an electrode according to any one of the variants described herein having the ability to reside in vivo in said subject for a limited period of time, such as the time of a surgery.

An electrode or a system according to any one of the embodiments described herein and in the claims may be used for example for the treatment, diagnosis, monitoring and/or prevention of signs and/or symptoms associated with pathological conditions affecting the nervous system, in particular the central and/or peripheral nervous system of a subject.

According to one aspect of the invention, said electrode or system may be used to electrically detect a physiological parameter or stimulate an electrical response in a target nerve of a subject. The detection or stimulation activity may be carried out as part of a therapeutic or preventive medical treatment (e.g. for diagnostic purposes) in a subject. Accordingly, a further aspect of the invention concerns the use of an electrode or a system according to the present description for detecting a physiological signal and/or stimulating an electrical and/or pharmacological activity of a structure of the tubular body, in particular a nerve, of a subject.

Advantageously, the use of the electrode or of the system according to the invention can be intended for the treatment of one or more pathological conditions. By way of example, the electrode or the system according to the present disclosure may be used for the treatment of one or more pathological conditions where detection or monitoring and/or stimulation of a nerve or other tubular structures of the body could be advantageous, and in applications relating to the treatment of pathological conditions of the peripheral nerves. An exemplary and non-limiting list of possible applications of an electrode as well as of a system according to the present invention comprises: the treatment of post-operative pain, the treatment of foot drop, the treatment of bladder incontinence, the treatment of obstructive sleep apnoea, the treatment of diabetes by stimulation of the hepatic vagus nerve, the treatment of erectile dysfunctions, stimulation of the phrenic nerve, treatment of intractable epilepsy or depression resistant to treatment by stimulation of the vagus nerve (VNS), the treatment of heart failure by stimulation of the vagus nerve (VNS), the treatment of drug-resistant hypertension by stimulation of the vagus nerve (VNS), the treatment of autoimmune diseases, sensory feedback and/or control with prosthetic limbs, recovery of mobility by stimulation of the motor nerves in patients suffering from paralysis or stroke, and stimulation and recording of the facial nerve for treatment of ischemic stroke or for the recovery of facial expressions.

A method for implanting an electrode according to any one of the embodiments illustrated in the present description and in the claims at a target nerve of a subject in need thereof is also described herein, the method comprising the following steps:
providing an electrode 100 or a system comprising it according to any one of the embodiments described herein;
winding said electrode around said target nerve so as to electrically connect said at least one portion 31 of the pattern 20 of the main body of the electrode with the outer surface of said target nerve; or, alternatively,
introducing said electrode into said target nerve so as to electrically connect said at least a portion 31 of the pattern 20 of the main body of the electrode with the inner surface of said target nerve.

Preferably, the previously described method further comprises at least one step of electrical connection of at least a portion 32 of said electrode 100 to an external device according to any one of the previously exemplified embodiments.

### EXAMPLES

Some non-limiting embodiments of the method according to the present invention are given herein for illustrative purposes.

### Example 1-Manufacture of an Implantable Neural Electrode According to a First Embodiment of the Invention

A first implantable neural electrode according to the invention was prepared by performing the following steps:

1) Realization of a CAD file representing the three-dimensional model of the electrode. The prototype made is a parallelepiped with the following dimensions: 50×15×0.15 mm. Once loaded into the software of the printer used (3D Bioplotter, Envisiontec), a slicing is carried out to define how many layers (insulating and conductive) must be printed by the machine. An insulating layer at the base, a layer of conductive traces (or conductive pattern), and an insulating layer at the end were then printed, in such a way as to leave exposed the traces in some points, which constitute the sensitive elements of the device.

2) Preparation of an ink composed of elastomeric insulating material. The material considered in this implementation is PDMS (Sylgard 184), in a 10:1 monomer: curing.cross-linking agent ratio. The two components are weighed and then mixed using a spatula, after which the compound is poured into a syringe to be able to be subsequently printed, and subsequently a vacuum pump degassing treatment is carried out for 20 minutes. Once removed from the vacuum pump, it is stored at 4-6° C. until the printing step.

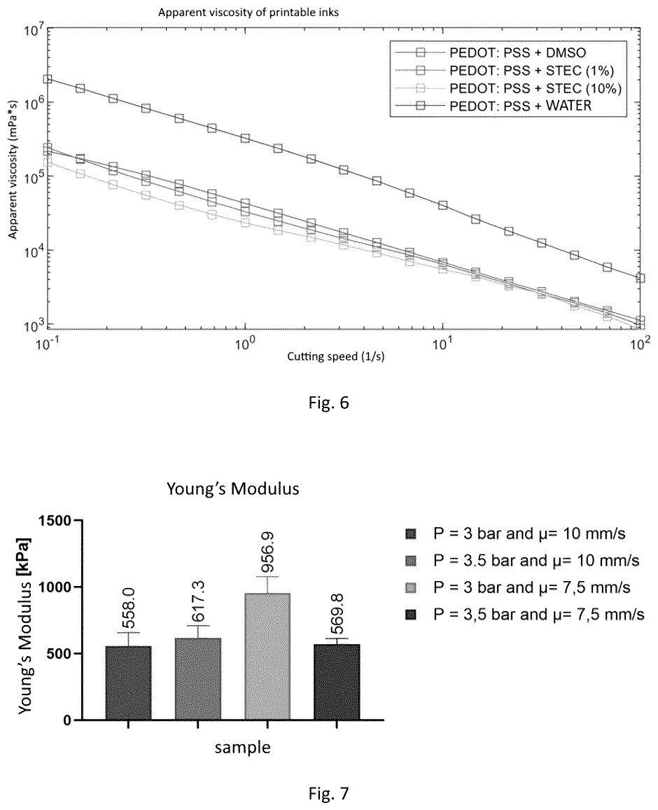

3) Preparation of an ink composed of conductive material. The material considered in this implementation is a solution composed of PEDOT:PSS, deionized water and dimethylsulfoxide (DMSO). In addition, additives, such as ionic surfactants, may be added to improve the conductive capabilities of the ink. The pure PEDOT:PSS is inserted into a custom mould composed of a Teflon cylinder placed between two metal discs. This structure is then placed in a freezer at a temperature of −80° C. for 24 h. The mould is made with metal discs at the ends to create a thermal gradient capable of creating a nanofibrillar structure of PEDOT:PSS, in order to be able to align the polymeric chains and increase conductivity. Once removed from the freezer, the frozen PEDOT:PSS is freeze-dried for 72 h. The structure thus obtained is redispersed in a solution of water and DMSO (80:20) with a concentration of 5-6% wt. The freeze-dried PEDOT:PSS is mixed with the solution of water and DMSO using an automatic mixer for 5 min, in order to obtain a homogeneous paste. In the case where an ionic surfactant is used, PEDOT:PSS is dispersed in a solution of water and ionic surfactant, with a concentration of surfactant equal to 1% wt. (FIG. 6). Subsequently, the ink is inserted into a syringe to be printed, and stored at 4-6° C. until the printing step.

4) Loading of the inks inside the printer. The syringes with the two inks (insulating and conductive) are loaded into the printer, securing them to the printheads with a mechanical locking mechanism. They are then connected to the compressed air line that allows the extrusion of the material.

5) Setting of the printing parameters typical of the extrusion process characteristic of the materials used. In an extrusion printing, the two main parameters are extrusion pressure and speed, as well as the temperature of the head. The following parameters are used for the PDMS:
a) speed 7.5-10 mm/s, pressure 3-3.5 bar, temperature 25° C. The following parameters are used for PEDOT:PSS:
b) speed 6 mm/s, pressure 0.8 bar, temperature 25° C. for the solution of PEDOT:PSS and DMSO.
c) speed 6 mm/s, pressure 1.2 bar, temperature 25° C. for the solution of PEDOT:PSS and ionic surfactant.

To speed up the PDMS polymerization process and perform a thermal annealing of the PEDOT:PSS ink, the printing surface is heated to 80° C. The thermal annealing process has a dual purpose: to allow complete evaporation of the solvent and to further increase the conductivity of the printed conductive ink. To allow complete polymerization of the PDMS and stabilization of the PEDOT:PSS immediately after printing, 1 min is awaited between one printing layer and the next.

6) Printing of the first flexible insulating layer. The first layer of PDMS is printed with a pattern directed along the largest size of the rectangle, until it is completely filled.

7) Printing of the conductive polymer traces. The traces of PEDOT:PSS are printed along the largest size of the underlying PDMS rectangle, with a width equal to 150 μm and a length equal to 45 mm, so as to have 2.5 mm at the two ends of PDMS free.

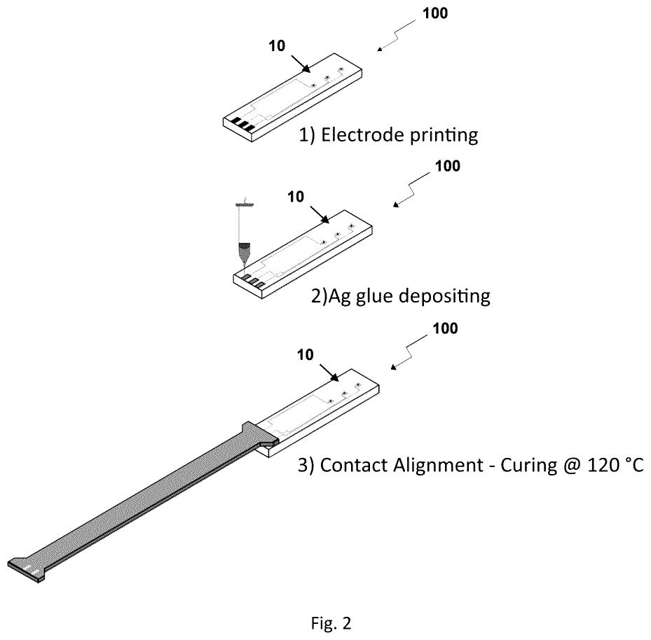

8) Printing of the second flexible insulating layer. The second layer of PDMS is made in such a way as to leave some parts of the traces in PEDOT:PSS exposed, which will be used as a sensitive part of the device, to record the nervous signal or to inject current. (FIG. 2). A prototype of the device made following this approach is shown in FIG. 5.

The device thus made was subsequently electrically connected to a printed circuit board (PCB) made specifically for the interface between the device itself and the external electronics. A diagram of the electronic connections is illustrated in FIG. 4 and comprises:
A flexible polyimide PCB connected by silver conductive glue to the conductive traces of the electrode at the end thereof.
The aforesaid flexible PCB connected to a rigid custom PCB, so that it can interface with external electronics (neural amplifier/stimulator).

The device made in the invention can be used in the field of neural interfaces both to record the electrical signal coming from the nerve, and to modulate the activities thereof by injecting electrical current. Since the design is highly customizable, this device can be made for extraneural applications, i.e. winding it around the nerve, or for intraneural applications, i.e. inserting the device inside the nerve, so as to reach the internal fascicles of the same, and greatly increase the recording/stimulation selectivity (FIG. 3).

1. An implantable neural electrode comprising a flexible main body extending between a first end and a second end, configured to be at least partially disposed around or inserted within a target nerve, said main body comprises:
a first electrically insulating layer;
a second electrically insulating layer; and
an electrically conductive pattern interposed between said first and second layers;
wherein said pattern comprises a first portion, optionally partially outwardly exposed, configured to electrically come into contact with said target nerve, and a second portion, optionally outwardly exposed, configured to electrically connect said electrode to an external device;
wherein said first layer and second layer comprise at least one polymeric material and said pattern comprises at least one conductive hydrogel or polymer, and wherein said first layer, said second layer, and said pattern do not contain metallic nanostructures or components.

2. The electrode according to claim 1, wherein said first layer, said second layer and said pattern are obtained from compositions having pseudo-elastic and/or thixotropic properties, and/or wherein said first layer, said second layer and said pattern are obtained by a printing process.

3. The electrode according to claim 1, wherein said first layer and said second layer consist of at least 80%, at least 90%, at least 95% or at least 98% by weight of said polymeric material and wherein said pattern consists of at least 80%, at least 90%, at least 95% or at least 98% by weight of said conductive hydrogel or polymer.

4. The electrode according to claim 1, wherein said polymeric material comprises at least one elastomeric polymer selected from silicone elastomers, thermoplastic elastomers, liquid crystal elastomers, elastomeric polyesters and polycarbonates, and mixtures thereof.

5. The electrode according to claim 1, wherein said polymeric material comprises at least one elastomeric polymer selected from polydimethylsiloxane (PDMS), polyurethane (PU), poly-ε-caprolactone (PCL) and mixtures thereof.

6. (canceled)

7. The electrode according to claim 1, wherein said polymeric material comprises one or more biodegradable polymeric materials selected from polypeptides, proteins, elastomeric polymers, gels or hydrogels of a gelling agent of natural origin, and mixtures thereof.

8. The electrode according to claim 7, wherein said polymeric material comprises or consists of fibroin.

9. The electrode according to claim 7, wherein said polymeric material comprises or consists of an elastomeric polymer selected from poly-hydroxyalkanoates (PHA), in particular poly(3-hydroxybutyrate), poly(4-hydroxybutyrate), poly(3-hydroxyvalerate), poly(3-hydroxyhexanoate), poly(3-hydroxyoctanoate), poly(3-hydroxydecanoate), polyurethanes (PU), in particular branched-chain PU, polyglycerol sebacate (PGS), copolymers and/or mixtures thereof.

10. The electrode according to claim 7, wherein said polymeric material comprises or consists of polyglycerol sebacate (PGS).

11. The electrode according to claim 7, wherein said polymeric material comprises or consists of alginate gel or gelatin of vegetable or animal origin.

12. The electrode according to claim 1, wherein said conductive hydrogel or polymer is selected from polyaniline, polypyrrole, poly(3,4-ethylenedioxythiophene) polystyrene sulfonate (PEDOT:PSS) and mixtures thereof.

13. The electrode according to claim 12, wherein said conductive polymer is PEDOT:PSS.

14. The electrode according to claim 1, wherein said conductive hydrogel comprises a mixture of a conductive polymer and an elastomer.

15. The electrode according to claim 1, wherein said at least one polymeric material is PDMS and said at least one conductive hydrogel or polymer is PEDOT:PSS.

16. The electrode according to claim 1, wherein said first layer, said second layer and said pattern further comprise at least one compound selected from thickening agents, surfactants, solvents and plasticizers.

17. The electrode according to claim 1, wherein said pattern comprises at least one ionic surfactant.

18. The electrode according to claim 1, wherein said pattern comprises a plurality of said first portions at least partially outwardly exposed and configured to electrically come into contact with said target nerve.

19. The electrode according to claim 1, wherein said at least one second portion of the pattern it is located at one end of the main body.

20. The electrode according to claim 1, wherein said pattern is in a form of strips, lines and/or points connected to each other.

21. The electrode according to claim 1, wherein said pattern is in a form of parallel strips spaced between each other.

22. The electrode according to claim 21, wherein said strips each have a width between 50 and 500 μm, or have the width equal to 150 μm, and a length between 10 and 200 mm, or have the length equal to 45 mm.

23. The electrode according to claim 1, wherein said first end and said second end of the main body are mutually engageable in such a way as to close and keep the electrode in a desired position.

24. The electrode according to claim 1, wherein said main body carries a slot at said first end, which slot is configured to receive the second end of the main body and allow sliding therein of said main body so as to close and maintain the electrode in a desired position.

25. The electrode according to claim 1, wherein said flexible main body is of a cuff type.

26. The electrode according to claim 1, wherein said main body has a thickness comprised between 0.01 and 1 mm, or equal to 0.15 mm.

27. A system comprising an electrode according to claim 1 operatively connected to an external device by said at least one second portion of the electrically conductive pattern.

28. (canceled)

29. (canceled)

30. (canceled)

31. A process for manufacturing of an implantable neural electrode comprising a flexible main body configured to be at least partially disposed around or inserted within a target nerve, said main body extending between a first end and a second end, which process comprises the following steps:
(i) providing a first composition comprising at least one electrically insulating polymeric material or precursors thereof, and a second composition comprising at least one electrically conductive hydrogel or polymer or precursors thereof, wherein said compositions have pseudo-elastic and/or thixotropic properties and do not contain metallic nanostructures or components;
(ii) depositing a layer of said first composition on a flat surface of a substrate, so as to form a first electrically insulating layer of said main body comprising said at least one polymeric material;
(iii) depositing a layer of said second composition onto the first layer obtained in step (ii) so as to form, onto said first layer, an electrically conductive pattern comprising said at least one conductive hydrogel or polymer;
(iv) depositing a layer of said first composition onto the pattern obtained in step (iii) so as to form, onto said pattern, a second electrically insulating layer of said main body comprising said at least one polymeric material, wherein said depositing is carried out in such a way that at least one first portion and at least one second portion of the pattern are left at least partially outwardly exposed;
wherein all depositing steps from (ii) to (iv) are carried out by means of an extrusion printing technique.

32-64. (canceled)
